(19) United States
(12) Patent Application Publication
(10) Pub. No.: US 2014/0200395 A1
(43) Pub. Date: Jul. 17, 2014

(54) APPARATUSES AND METHODS FOR PREVENTING OR REVERSING HEART DILATION

(71) Applicants: Bilal Shafi, Philadelphia, PA (US); Karen Havenstrite, San Francisco, CA (US); Ryan Krone, Oakland, CA (US)

(72) Inventors: Bilal Shafi, Philadelphia, PA (US); Karen Havenstrite, San Francisco, CA (US); Ryan Krone, Oakland, CA (US)

(21) Appl. No.: 14/103,748

(22) Filed: Dec. 11, 2013

Related U.S. Application Data
(60) Provisional application No. 61/735,935, filed on Dec. 11, 2012.

(51) Int. Cl. A61L 31/14; A61L 31/06

(52) U.S. Cl. CPC A61L 31/145; A61L 31/06
USPC 600/37

(57) ABSTRACT

A method of delivering a material inside a patient includes: inserting a distal end of an elongated device inside a patient next to a heart; deploying a sheath around at least a part of the heart using the elongated device; and delivering the material into a space between the heart and the sheath. A method of delivering a material inside a patient includes: inserting the material inside a body of the patient, wherein the material is in solid form, and has a rolled-up configuration; un-rolling the material; and placing the material around a heart of the patient so that the material wraps at least partially around the heart.

### RELATED APPLICATION DATA

This application claims priority to and the benefit of U.S. Provisional Patent Application No. 61/735,935, filed on Dec. 11, 2012, pending. The entire disclosure of the above application is expressly incorporated by reference herein.

### FIELD

This application relates to apparatus and methods for preventing or reversing heart dilation.

### BACKGROUND

Existing heart failure therapies are designed to treat an already damaged heart. These heart failure therapies include implants inserted into the damaged heart that isolate the non-functional muscle segment from the functional segment, which decreases the overall volume that the heart has to pump. Other heart failure therapies include polymer implants that are placed inside a muscle of the heart for reshaping of the heart. Yet another heart failure therapy involves surgical ventricular reconstruction, which helps the heart improve its ability to pump blood effectively. Cardiac defibrillators are another heart failure therapy that constantly monitor the rate and rhythm of the heart, and deliver therapies by way of an electrical shock. Left ventricular assist devices are mechanical pumps used to support heart function in people who have experienced heart failure. Lastly, medications may be prescribed by surgeons to patients to deal with heart failure conditions.

The above existing therapies target patients with already existing or end stage heart failure. Such therapies may be invasive, immune responsive, and difficult and expensive to implement. For the foregoing reasons, applicant of the subject application determines that it would be desirable to have new apparatuses and methods for preventing heart dilation.

### SUMMARY

In accordance with some embodiments, a method of delivering a material inside a patient includes: inserting a distal end of an elongated device inside a patient next to a heart; deploying a sheath around at least a part of the heart using the elongated device; and delivering the material into a space between the heart and the sheath.

In accordance with other embodiments, a method of delivering a material inside a patient includes: inserting the material inside a body of the patient, wherein the material is in solid form, and has a rolled-up configuration; un-rolling the material; and placing the material around a heart of the patient so that the material wraps at least partially around the heart.

In accordance with other embodiments, a medical device includes: an elongated tube having a proximal end and a distal end, and a body extending between the proximal end and the distal end, wherein the elongated tube further comprises a lumen extending between the proximal end and the distal end; a deformable sheath coupled to the distal end of the elongated tube; a member moveable relative to the elongated tube for changing the deformable sheath from a confined configuration to a deployed configuration; and a delivery tube located in the elongated tube, wherein the delivery tube is in fluid communication with an opening at the deformable sheath.

In accordance with other embodiments, a medical device includes: an elastic container having a first end, a second end, and a body between the first end and the second end, wherein the first end has an opening, and the second end is closed;

wherein the elastic container has an un-deployed configuration and a deployed configuration; and wherein when the elastic container is in the deployed configuration, the elastic container has a size and a shape suitable for wrapping around at least a part of a heart.

In accordance with other embodiments, a medical method includes: delivering a 8-arm PEG-vinyl sulfone into a device; delivering a 4-arm PEG-thiol into the device; and forming a cross-linked structure using the 8-arm PEG-vinyl sulfone and the 4-arm PEG-thiol; wherein the structure is applied to a surface of a heart for preventing an undesirable dilation of the heart.

Other and further aspects and features will be evident from reading the following detailed description of the embodiments.

### DESCRIPTION OF THE DRAWING FIGURES

The drawings illustrate the design and utility of embodiments, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict exemplary embodiments and are not therefore to be considered limiting in the scope of the claims.

FIGS. 1A-1C illustrates a device for treating a heart in accordance with some embodiments.

FIG. 2 illustrates an elongated tube with multiple lumens in accordance with some embodiments.

FIGS. 3A-3D illustrate a process of using the device of FIG. 1A in accordance with some embodiments.

FIG. 4 illustrates an example of a material that may be delivered onto the heart.

FIG. 5 illustrates a relationship between backpressure being applied and polymeric concentration.

FIGS. 6A-6B illustrate a medical device for treating a heart in accordance with other embodiments.

FIGS. 7A-7C illustrate a process of using the device of FIG. 6 in accordance with some embodiments.

### DETAILED DESCRIPTION

Various embodiments are described hereinafter with reference to the figures. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the claimed invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

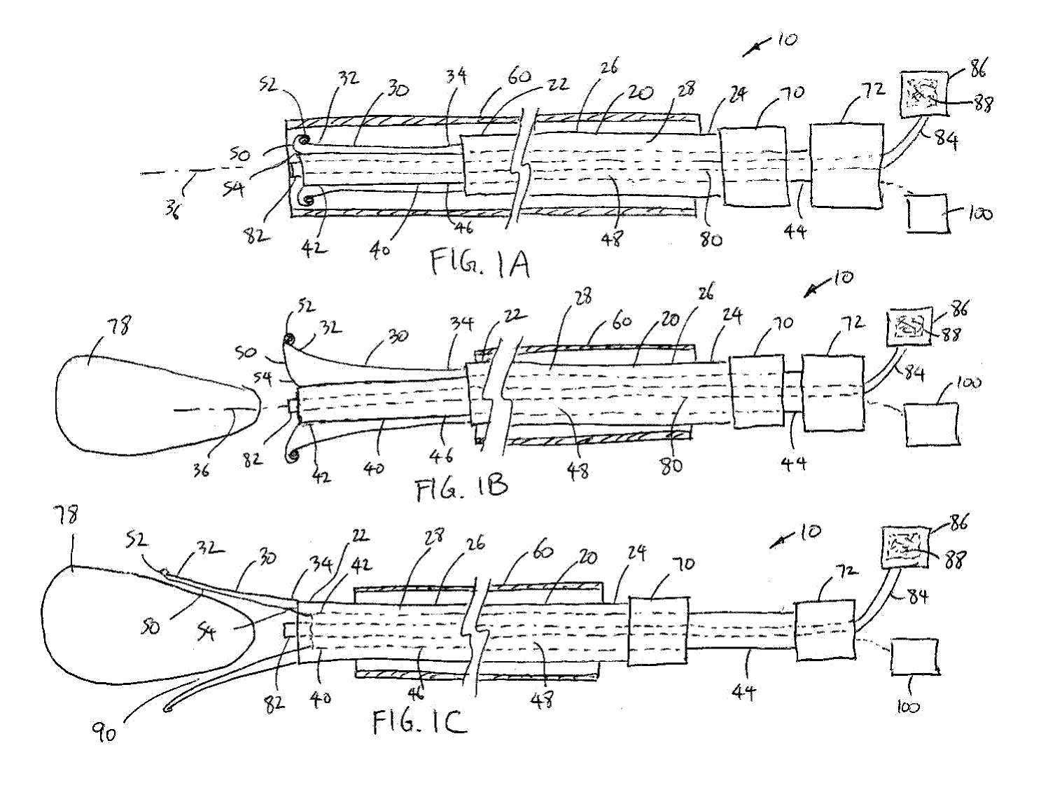

FIGS. 1A-1C illustrate a device 10 for treating a heart that involves applying a material onto the heart in accordance with some embodiments. The device 10 includes a first elongated tube 20, a support structure 30 coupled to the first elongated tube 20, a second elongated tube 40 slidably disposed in the first elongated tube 20, and an elastic sheath 50 with one end coupled to the support structure 30, and another end coupled to the second elongated tube 40. The device further includes an exterior tube 60 for housing the first and second elongated tubes 20, 40, the support structure 30, and the sheath 50.

As shown in the figure, the first elongated tube 20 has a distal end 22, a proximal end 24, a body 26 extending between the distal end 22 and the proximal end 24, and a lumen 28 in the body 26. In some embodiments, the first elongated tube 20 may be rigid. In other embodiments, the first elongated tube 20 may be flexible.

The support structure 30 has a distal end 32 and a proximal end 34. In the illustrated embodiments, the proximal end 34 of the support structure 30 is coupled to the distal end 22 of the first elongated tube 20, and the distal end 32 of the support structure 30 is coupled to the sheath 50. In some embodiments, the support structure 30 may include a plurality of elastic supports, such as metallic wires. The support structure 30 may have a confined configuration when housed inside the exterior tube 60, and may expand radially from a longitudinal axis 36 of the device 10 when deployed out of the exterior tube 60. In some embodiments, the support structure 30 may have a length along the axis 36 of the device 10 that is equal to, or longer than, a length of the heart being treated. Such configuration allows the support structure 30 to be deployed around the entire length of the heart. In other embodiments, the support structure 30 may have a length along the axis 36 of the device 10 that is less than a length of the heart being treated. Such configuration allows the support structure 30 to be deployed around a portion of the entire length of the heart.

The second elongated tube 40 has a distal end 42, a proximal end 44, a body 46 extending between the distal end 42 and the proximal end 44, and a lumen 46 in the body 46. The distal end 42 of the second elongated tube 40 is coupled to the sheath 50. As shown in the figure, the device 10 further includes a first handle 70 coupled to the proximal end 24 of the first elongated tube 20, and a second handle 72 coupled to the proximal end 44 of the second elongated tube 40. Relative positioning between the first and second elongated tubes 20, 40 may be achieved by manipulating the first handle 70, the second handle 72, or both.

The elastic sheath 50 has a first end 52, a second end 54, a body 55 extending between the first end 52 and the second end 54, a first opening 56 at the first end 52, and a second opening 58 at the second end 54. The first end 52 of the elastic sheath 50 is coupled to the distal end 32 of the support structure 30, and the second end 54 of the elastic sheath 50 is coupled to the distal end 42 of the second elongated tube 40. As shown in the illustrated embodiments, the sheath 50 is rolled-up at the distal end 32 of the support structure 30, and may be un-rolled by moving the first handle 70 distally relative to the second handle 72, or by moving the second handle 72 proximally relative to the first handle 70. In other embodiments, the sheath 50 may be rolled-up at the distal end 42 of the second elongated tube 40. In some embodiments, the sheath 50 may be made of a biocompatible material (e.g. elastic silicone rubber).

As shown in FIG. 1A, before the sheath 50 is deployed, the sheath 50 is rolled-up, and the sheath 50 and the support structure 30 are in a confined configuration inside the exterior tube 60. During use, the sheath 50 and the support structure 30 may be deployed out of the exterior tube 60 (FIG. 1B). As shown in the figure, after the support structure 30 is deployed out of the tube 60, the support structure 30 expands radially away from the axis 36. In some embodiments, the expanded support structure 30 forms a distal opening having a size and a shape that are suitable for placement around the heart 78. After the sheath 50 and the support structure 30 are deployed out of the exterior tube 60, the first and second elongated tubes 20, 40 may be manipulated to un-roll the sheath 50 into a deployed configuration (FIG. 1C). In some embodiments, the distal end 42 of the second elongated tube 40 may be held stationary relative to the heart 78, and the first elongated tube 20 carrying the support structure 30 may be translated distally relative to the second elongated tube 40. Such action pushes the end 52 of the sheath 50 away from the end 54, thereby un-rolling the sheath 50. In other embodiments, the first elongated tube 20 carrying the support structure 30 may be held stationary relative to the heart 78, and the second elongated tube 40 may be translated proximally relative to the first elongated tube 20. Such action pulls the end 54 of the sheath 50 away from the end 52, thereby un-rolling the sheath 50. When in the deployed configuration, the sheath 50 has a cup-shape with a size and a shape that are suitable for placement around at least a part of a heart 78. As shown in the figure, the first opening 56 at the first end 52 of the sheath 50 allows part of the heart 78 to enter therethrough.

In other embodiments, instead of the rolled-up configuration, the sheath 50 may be folded, unstretched, or collapsed into a low profile suitable for delivery of the sheath 50, and the sheath 50 may be unfolded, stretched, or expanded into a deployed configuration for covering the heart 78.

As shown in the illustrated embodiments, the device 10 further includes a delivery tube 80 having a distal end 82 and a proximal end 84. The proximal end 84 of the delivery tube 80 is coupled to a source 86 of material 88. The delivery tube 80 is disposed within the lumen 48 of the second elongated tube 40. In other embodiments, the delivery tube 80 and/or the source 86 of material 88 is not a part of the device 10. In such cases, during use, the delivery tube 80 is inserted into the second elongated tube 40 of the device 10, and the source 86 of material 88 is coupled to the second end 84 of the delivery tube 80. The delivery tube 80 is configured to deliver the material 88 from the source 86 to a space 90 that is between the heart 78 and the sheath 50. The source 86 may be a mechanical pump, a syringe, or any of other types of devices that is capable of supplying fluid, in different embodiments.

In some embodiments, the delivery tube 80 has a single lumen for delivering the material 88 from the source 86. In other embodiments, the delivery tube 80 may have a first lumen and a second lumen, and the source 86 may have a first compartment for housing a first component of the material 88 and a second compartment for housing a second component of the material 88. In such cases, the first and second lumens of the delivery tube 80 are configured to deliver the first and second components of the material 88, respectively. In some embodiments, the first and second components of the material 88 are not combined until they are delivered out of the distal end 82 of the tube 80 into the space 90 between the heart 78 and the sheath 50. In other embodiments, the distal end 82 of the tube 80 may be located inside the second elongated tube 40 so that the distal end 82 is proximal to the distal end 42 of the second elongated tube 40. In such cases, the first and second components of the material 88 are delivered into the lumen 48 of the second elongated tube 40 in which the components of the material 88 are combined before the material 88 is transmitted out of the distal end 42 of the second elongated tube 40.

In further embodiments, the source 86 may have more than two components (e.g., three or more components) for the material 88 that are stored in separate respective compartments.

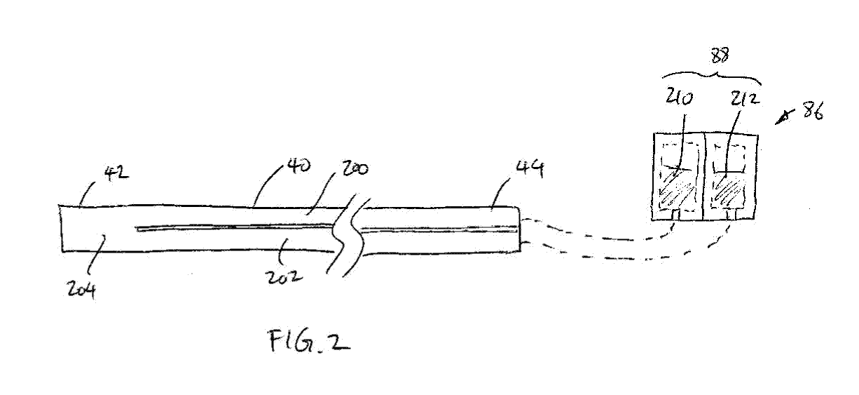

Also, in other embodiments, the deliver tube 80 is not required. Instead, the second elongated tube 40 may function as a delivery channel for delivering the material 88 from the source 86 to the space 90 that is between the heart 78 and the sheath 50. In some embodiments, the lumen 48 of the second elongated tube 40 is used for delivering the material 88 from the source 86. In other embodiments, the tube 40 may have an additional (a second) lumen, and the source 86 may have a first compartment for housing a first component of the material 88 and a second compartment for housing a second component of the material 88. In such cases, the first and second lumens of the second elongated tube 40 are configured to deliver the first and second components of the material 88, respectively. In some embodiments, the first and second components of the material 88 are not combined until they are delivered out of the distal end 42 of the second elongated tube 40 into the space 90 between the heart 78 and the sheath 50. In other embodiments, the second elongated tube 40 may include first and second lumens 200, 202 that do not extend all the way to the distal end 42 (FIG. 2). Instead, the lumens 200, 202 extend to a position that is proximal from the distal end 42 of the tube 40. In such cases, as shown in FIG. 2, the first and second components of the material 88 are delivered into a lumen 204 that is distal to the lumens 200, 202, in which the components of the material 88 are combined before the material 88 is transmitted out of the distal end 42 of the second elongated tube 40.

Returning to FIG. 1A, in some embodiments, the device 10 may optionally further include a suction device 100 for applying suction at the space 90 between the heart 78 and the sheath 50 to remove air pockets. In some embodiments, the second elongated tube 40 may be used to apply the suction. In other embodiments, the device 10 may include a separate tube coupled to the suction device 100 for applying the suction. The suction tube may be placed in the second elongated tube 40. For example, the second elongated tube 40 may have an additional lumen dedicated for applying suction. In other embodiments, the suction tube may be placed next to the second elongated tube 40. In such cases, the sheath 50 may have an opening at the body 55 that is coupled to the suction tube. Such configuration allows suction to be applied through the opening at the body 55 of the sheath 50.

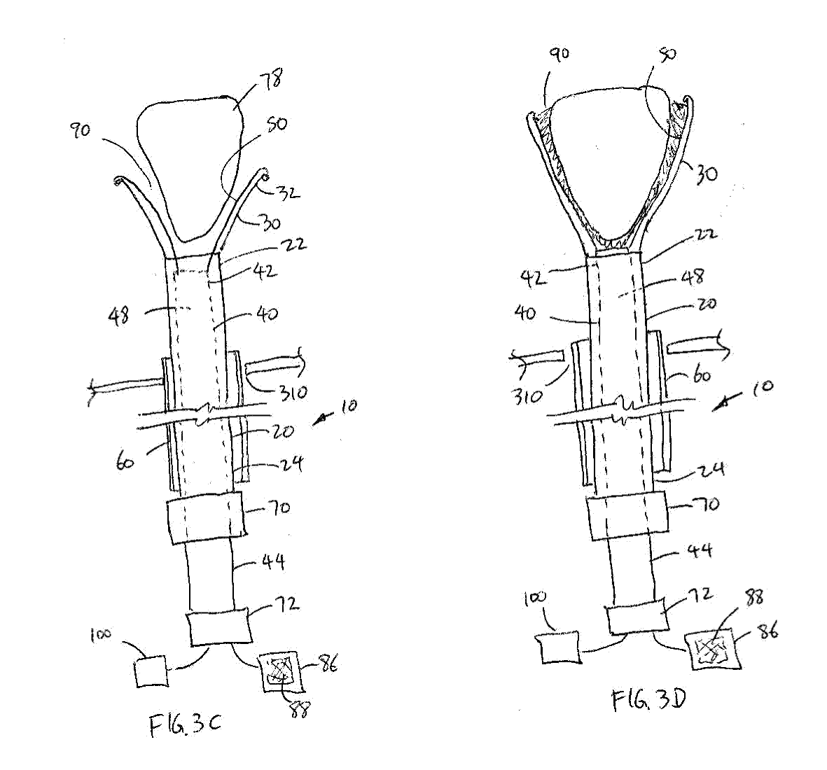

In some embodiments, the material 88 may be polymerizing hydrogel. Also, in some embodiments, the material 88 may be a poly(ethylene glycol) (PEG) based hydrogel. FIG. 4 illustrates an example of a polymerizing hydrogel that may be used. As shown in the figure, the material 88 is formed from a 8-arm, PEG-vinyl sulfone component 400, and a 4-arm PEG-thiol component 402, which when combined, form a cross-linked structure 404. In some embodiments, the PEG-vinyl sulfone may be a 10 kDa PEG-vinyl sulfone, and the PEG-thiol may be a 10 kDa PEG-thiol. In other embodiments, the material 88 may have other components. The molar ratio of thiol groups to vinyl sulfone groups may be 1:1 in some embodiments. When the two components are mixed together in a buffer at physiologic temperature (e.g. 37C.°) and physiological pH (e.g. pH 7, pH8, etc.), they rapidly form a cross-linked structure. The linking chemistry is a Michael-type conjugate-addition reaction that requires no additional energy input to initiate the polymerization (e.g. no light or no toxic initiators) and produces no toxic byproducts in the reaction process. Also, in some embodiments, the polymerizing hydrogel may polymerize at a ratio of 2:1 in weight for the thiol and vinyl sulfone groups, respectively.

In some embodiments, the material 88 may be tunable within a range that is appropriate for cardiac passive restraint so the mechanical strength may be adjusted to the desired level in a predictable and reproducible manner. For example, by changing the functionality of the cross-linking PEG thiol from a 4-arm to a 2-arm PEG-thiol, the network structure may less densely cross-linked and therefore weaker. If the cross-linker is changed to an 8-arm PEG-thiol, the resulting network structure may be more densely cross-linked and stronger. For this reason, the reaction is more controlled, giving a more reproducible strength. In some embodiments, the material strength of the material 88 may be altered simply by adjusting the polymer concentration in the precursor solution. Since the relationship between strength and concentration is linear, the strength is predictably adjusted by changing the concentration in the precursor solution.

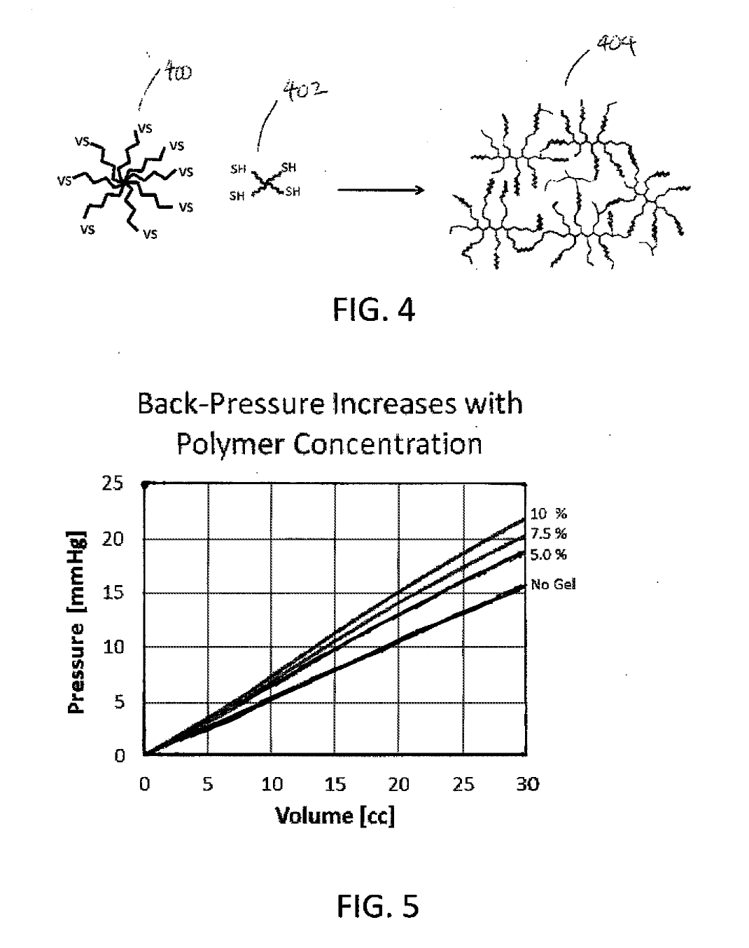

In some embodiments, the material 88 forms a biochemically “blank” extracellular matrix structure around the heart muscle that provides mechanical support for a period between 1 and 12 months, and more preferably between 5 and 7 months, such as 6 months, before the material 88 degrades. The purpose of the matrix structure is prevent dilation of the heart to allow the heart to heal, while not completely constricting the heart to allow cardiac output. In some embodiments, the matrix structure provides a backpressure on the heart with a therapeutic effect and a preserved cardiac output that is anywhere between 1 and 5 mmHg, and more preferably between 2 and 4 mmHg, such as 3 mmHg. In some embodiments, the mechanical strength of the material 88 may be adjusted to further elucidate the role of wall stress in triggering biochemical and neurochemical cascades that ultimately lead to left ventricle (LV) remodeling and heart failure. Allowing the material 88 to be tunable is desirable because it enables the user or the provider of the material 78 to adjust the cardiac restraint device to achieve a precisely controlled level of support. As discussed, the material strength may be adjusted by altering the polymeric concentration. This in turn, would result in a change of the pressure being applied by the matrix structure against the heart. FIG. 5 illustrates a relationship between back pressure being applied by the matrix structure against the heart and polymeric concentration. As shown in the figure, the higher the polymeric concentration, the higher the back pressure will be applied by the matrix structure.

In some embodiments, the molecular weight of the components may be anywhere between 5 kDa and 15 kDa, and more preferably between 9 kDa and 11 kDa, such as 10 kDa, which allows for degradation of the material 88 that can be safely eliminated by the body. The degradation of the material 88 may be adjusted by changing the bonds that link the network together. For example, a controlled degradation may be achieved by cross-linking the network with an enzymatically-degradable peptide sequence that is flanked by thiol containing cysteines on each end. In some embodiments, depending upon the peptide sequence, the material 88 may degrade on demand by injecting the enzyme into the pericardial space where the material 88 is adhered to. In other embodiments, the material 88 may be degraded using different cross-linking bonds including oximes (reaction between aldehyde and hydroxyl amine).

In some embodiments, the material 88 may be delivered to the pericardial space as a two component liquid that reacts rapidly in situ to form a cross-linked structure of poly(ethylene glycol) (PEG). The liquid based delivery of the material 88 allows the material 88 to be delivered in a minimally-invasive manner.

Also, in some embodiments, the material 88 may include cross-linked structure of hydrophilic polymers, and may be highly hydrated (e.g., >75% water, and more preferably, >90% water). Thus, the material 88 may be non-immunogenic and the resulting structure may mimic that of normal tissue.

In addition, in some embodiments, the material 88 does not swell after polymerization, thus decreasing the risk of the material breaking or shearing off from the heart. The ability for the material 88 to limit swelling in some embodiments is an advance from most hydrogels which typically absorb water after solidifying and increase by several times their initial size. The cross-linking chemistry of the material 88 may also provide adhesion to the epicardial surface of the heart in some embodiments. During polymerization, the material 88 is covalently linked to amines and thiols present on extracellular matrix proteins.

Furthermore, in some embodiments, hydrolysis of the material 88 may occur at thio-ether bonds, the original sites of cross-linking, and the hydrolysis may occur anywhere between 1 and 12 months, and more preferably between 5 and 7 months, such as 6 months.

Also, in some embodiments, the material 88 may have a hyperelastic behavior, in that it has less mechanical resistance (i.e. stiffness) in normal heart expansion, anywhere between 1 and 15% strain, and more preferably between 1 and 10% strain, and has increased stiffness at larger displacements that are greater than 10% strain for preventing undesirable heart dilation.

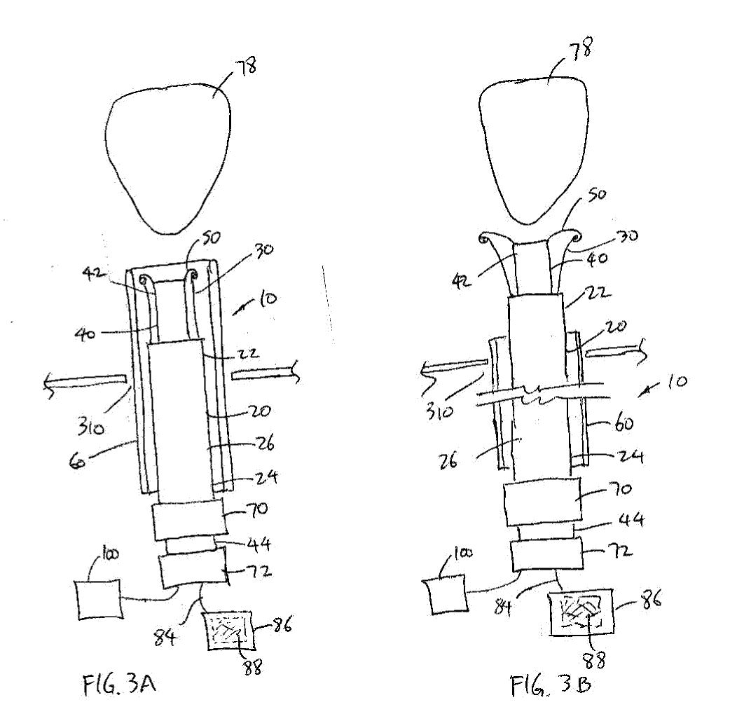

Having described various embodiments of the device 10, a method will now be described with reference to the device 10. FIGS. 3A-3D illustrate a method of using the device 10 of FIG. 1 to deliver the material 88 onto the heart 78 in accordance with some embodiments. First, as shown in FIG. 3A, a surgeon may create an incision 310 on a patient's skin. For example, the incision 310 may be created through a left thoracotomy, approaching through the 3rd, 4th and 5th intercostal spaces. In other embodiments, the incision 310 may be created in other locations on the patient's body. The surgeon may insert the distal end of exterior tube 60 through the incision 310 such that the device 10 is positioned at an apex of the heart 78. Alternatively, the exterior tube 60 may not be needed. Instead, the first elongated tube 20 housing the second elongated tube 40 may be inserted into the incision 310, without using the exterior tube 60.

Next, as shown in FIG. 3B, the support structure 30 and the sheath 50 may be deployed out of the tube 60. Such may be accomplished by translating the tube 60 proximally relative to the first elongated tube 20. Alternatively, such may be accomplished by translating the first elongated tube 20 distally relative to the tube 60.

Next, the sheath 50 is un-rolled to form a cup-configuration so that the sheath 50 surrounds at least a portion of the heart 78 (FIG. 3C). Such may be accomplished by moving the handle 70 distally relative to the handle 72 to thereby push the distal end 32 of the support structure 30 away from the distal end 42 of the second elongated tube 40. Alternatively, such may be accomplished by moving the handle 72 proximally relative to the handle 70 to thereby pull the distal end 42 of the second elongated tube 40 relative to the support structure 30.

In some embodiments, the deployed sheath 50 may cover an entire length of the heart 78. In other embodiments, the deployed sheath 50 may cover a portion of the length of the heart 78. The handle 70, and/or handle 72 may be manipulated to control an amount of sheath material being un-rolled, thereby controlling the depth of the cup-like deployed sheath 50. This allows a desired amount of the heart 78 to be surrounded by the deployed sheath 50.

In some embodiments, if the device 10 includes the suction device 100, the suction device 100 may be actuated to apply suction at the space 90 between the heart 78 and the sheath 50, to remove air pockets. The suction force may be applied using the lumen 48 of the second elongated tube 40. For example, the lumen 48 itself may transmit the suction force. In another example, a suction tube connected to the suction device 100 may be placed in the lumen 48 of the second elongated tube 40. In such cases, the suction tube is used (and the lumen 48 of the second elongated tube 40 is indirectly used) to transmit the suction force.

Refer now to FIG. 3D, next, the surgeon may operate the source 86 to deliver the material 88 from the source 88 into the space 90 between the heart 78 and the sheath 50. In some embodiments, the source 86 may include one or more syringes, in which cases, the material 88 may be delivered by operating the syringe(s). In other embodiments, the source 86 may include a mechanical pump for pumping one or more components of the material 88. In such cases, the mechanical pump may be activated to deliver the material 88.

In the illustrated embodiments, the second elongated tube 40 may be used (either directly or indirectly) to deliver the material 88 to the space 90. For example, if the device 10 includes the delivery tube 80, the delivery tube 80 may be used to deliver the material 88. In such cases, the delivery tube 80 is placed in the lumen 48 of the second elongated tube 40, and the lumen 48 then indirectly delivers the material 88 to the space 90. In other embodiments, if the device 10 does not include the delivery tube 80, then the lumen 48 of the second elongated tube 40 may itself be used to deliver the material 88 from the source 86.

As discussed, in some embodiments, the material 88 may be pre-mixed before being delivered from the source 86. In other embodiments, the material 88 may have multiple components (e.g., two components) that are combined after they are delivered from separate respective compartments of the source 86. The combining of the components of the material 88 may occur while the components are in the device 10, or after the components have been delivered into the space 90 between the heart 78 and the sheath 50. In some embodiments, the material 88 may include a 8-arm, 10 kDa PEG-vinyl sulfone component, and a 4-arm, 10 kDa PEG-thiol component, which when combined, form a cross-linked structure.

In some embodiments, during the delivery process of the material 88, an imaging device may be used to monitor an amount of the material 88 being delivered. For example, in some embodiments, the device 10 may optionally further include a camera for allowing the surgeon to view the delivery of the material 88 in situ. In other embodiments, the material 88 may include a contrast agent that is visible by medical imaging.

After the material 88 is delivered into the space 90, the sheath 50 functions as a container that contains the material 88 in fluid or gel form, until the material 88 solidifies. In particular, the material 88 will solidify with passage of time, thereby forming a layer around at least a part of the heart 78. For example, the components of the material 88 may polymerize in situ around the heart 78 (or part of the heart 78) anywhere between 5 seconds and 10 minutes, and more preferably between 10 seconds and 1 minute, such as 30 seconds. The formed layer around the heart 78 provides an elastic container that functions as reinforcement to prevent the heart 78 form dilation. For example, the formed layer may provide a backpressure onto the epicardial surface of the heart 78, hence reducing the local wall stress in the infarct region and preventing left ventricular remodeling. In some embodiments, the layer may be applied before the heart dilation starts to occur, thereby providing a preventive measure. In other embodiments, the layer may be applied after the heart dilation has started to occur. In such cases, the layer may prevent further heart dilation from occurring. Also, in some embodiments, the layer acts as a passive constraint for preventing heart dilation. In other embodiments, the layer may act as an active constraint for actively applying a compression pressure against the heart to thereby reverse the heart dilation.

In some embodiments, the formed layer from the material 88 itself adheres to the surface of the heart 78. In other embodiments, tissue reactive components, such as maleimides, aldehydes, or succinimyl esters that form links between the hydrogel and the heart 78, may be used to increase adhesion between the material 88 and the heart 78. Such tissue reactive component(s) may be applied to the surface of the heart 78 before the material 88 is applied onto the heart 78.

After the material 88 has solidified, the surgeon may then remove the device 10 from the patient. In particular, the sheath 50 may be rolled-up by moving the handle 70 towards the handle 72, or vice versa. Then the rolled-up sheath 50 and the support structure 30 may be retracted into the exterior tube 60, and the exterior tube 60 may then be removed. If the device 10 does not include the exterior tube 60, the first elongated tube 20 carrying the second elongated tube 40, the support structure 30, and the sheath 50 may then be directly removed from the patient.

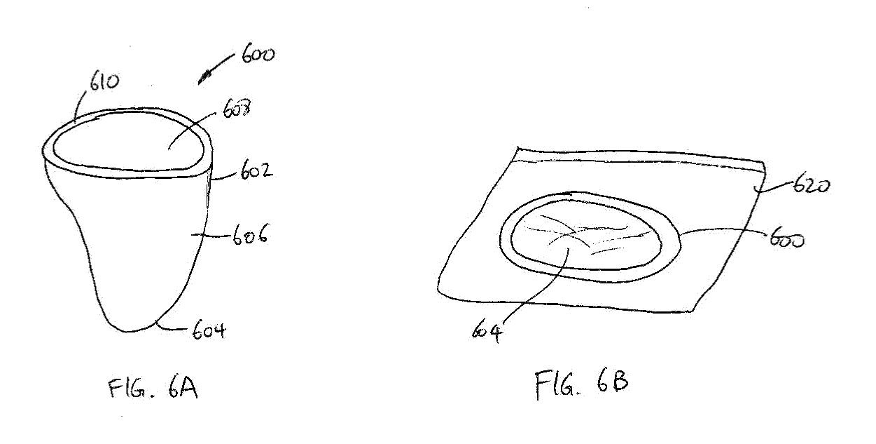

In the above embodiments, the material 88 is delivered in liquid or gel form into the space 90 between the heart 78 and the sheath 50, and the material 88 then solidifies to form a layer/container around the heart 78. In other embodiments, the container may be pre-formed. FIG. 6A illustrates a device 600 for treating the heart that involves applying a preformed material onto the heart in accordance with some embodiments. The device 600 is a container that includes a first end 602, a second end 604, a body extending between the first end 602 and the second end 604, and an opening 608 at the first end 602.

The device 600 also includes an elastic ring 610 at the first end 602 that corresponds with the opening 608. The elastic ring 610 is configured to help secure the device 600 relative to the heart, and may provide some stiffness for the edge at the first end 602 of the device 600. In some embodiments, the ring 610 may be enclosed in the body 606 of the device 600. In other embodiments, the ring 610 may be coupled to an exterior surface of the body 606. Also, in some embodiments, the ring 610 may be detachably coupled to the body 606 of the device 600.

In other embodiments, the device 300 does not have the ring 610. Also, in further embodiments, the device 600 may have an adhesive material instead of, or in addition to, the ring 610, to help secure the device 600 relative to the heart.

The container 600 is elastic, and has a size and a shape that are suitable for placement around at least a part of the heart. In some embodiments, the device 600 or the lumen 608 of the device 600 is shaped like a heart. This reduces or eliminates air space between the device 600 and the heart when the device 600 is placed around the heart. In some embodiments, the container 600 may have a shape and a size, and material composition that are configured to apply pressure towards the surface of the heart. Also, in some embodiments, the size, shape, and material composition of the container 600 may be customized for individual patient. For example, an image (e.g., a volumetric CT image) of the heart for a particular patient may be obtained, and the image may be used to customize the size, shape, and/or material composition of the container 600, so that when the container 600 is placed around the patient's heart, the container 600 will apply a certain desired amount of pressure against the heart. As shown in FIG. 6B, in some embodiments, the device 100 may be provided in a rolled-up configuration, and may be stored in a packaging 620.

The device 600 may be made from any of the materials described herein. For example, in some embodiments, the device 600 may include a polymerizing hydrogel. Also, in some embodiments, the device 600 may be made from a poly(ethylene glycol) (PEG) based hydrogel. In some cases, the material used to form the device 600 may include two components: a 8-arm, 10 kDa PEG-vinyl sulfone, and a 4-arm, 10 kDa PEG-thiol. The molar ratio of thiol groups to vinyl sulfone groups may be 1:1 in some embodiments. In other embodiments, the device 600 may be made from other materials.

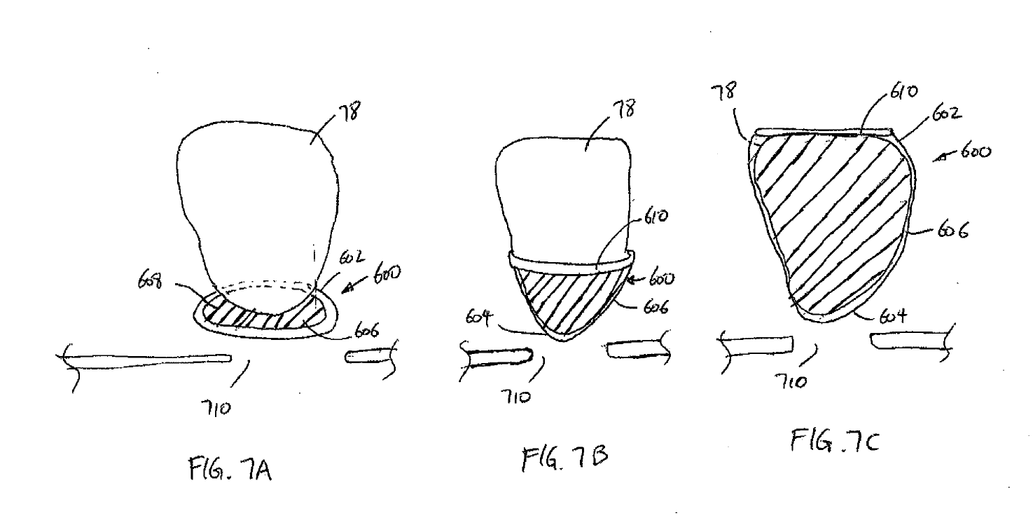

FIG. 7A-7C illustrates a method of using the device 600 of FIG. 6 to treat a heart in accordance with some embodiments. First, the surgeon creates an opening 710 through the patient's skin as in an open surgery, and the surgeon then manually places the opening 608 at the first end 602 of the device 600 around the heart 78 (FIG. 7A). In some embodiments, the device 600 may be placed around an apex of heart 78. In other embodiments, the device 600 may be placed around other locations of the hart 78.

As shown in FIG. 7B, next, the surgeon unrolls the device 600 starting from the apex of heart 78 towards the opposite end of heart 78. In some embodiments, the surgeon may hold onto the ring 610 of the device 600, and move the ring 610 along the heart 78 to thereby un-roll the device 600. The ring 610 is elastic and therefore may accommodate the different cross sectional dimensions of the heart 78 as the ring 610 around the heart 78 is moved along the heart 78.

In some embodiments, the body 606 of the device 600 adheres to the surface of the heart 78. In other embodiments, the ring 610 may also assist in securing the device 600 relative to the heart 78, like a rubber band. In other embodiments, an agent may be applied to the surface of the heart 78, or to the surface of the body 606, or to both, to increase adhesion between the body 606 of the device and the heart 78. In some embodiments, such agent is included with the device 600, and is already applied onto the body 606 when the device 600 is provided to the surgeon.

As shown in FIG. 7C, the device 600 is fully un-rolled in a deployed configuration. In this configuration, the device 600 completely covers and adheres to the heart 78. In other embodiments, the device 600 may be sized so that it covers only a part of the heart 78. In the illustrated embodiments, when the device 600 is in a fully deployed configuration, the first end 602 is located at one end of the heart 78, and the second end 604 is located at the apex of the heart 78. The ring 610 of the device 610 remains at one end of the heart 78. In other embodiments, the ring 610 of the device 600 may be detached from the body 606 after the device 600 has been deployed.

After the device 600 is deployed around the heart 78, the device 600 provides an elastic container that functions as reinforcement to prevent the heart 78 form dilation. For example, the device 600 may provide a backpressure onto the epicardial surface of the heart 78, hence reducing the local wall stress in the infarct region and preventing left ventricular remodeling. In some embodiments, the device 600 may be applied before the heart dilation starts to occur, thereby providing a preventive measure. In other embodiments, the device 600 may be applied after the heart dilation has started to occur. In such cases, the device 600 may prevent further heart dilation from occurring. Also, in some embodiments, the device 600 acts as a passive constraint for preventing heart dilation. In other embodiments, the device 600 may act as an active constraint for actively applying a compression pressure against the heart to thereby reverse the heart dilation.

In the above embodiments, the device 600 is described as being applied onto the heart manually by a surgeon. In other embodiments, the device 600 may be applied onto the heart percutaneously using a device. For example, in other embodiments, an elongated delivery device (e.g., a delivery catheter, a tube, etc.) may be provided, wherein the device 600 is detachably coupled to a distal end of the elongated delivery device. The delivery device may be configured to place the ring 610 around the heart, and may include an actuator that is configured to push the device 600 (when in the rolled-up configuration) so that the ring 610 moves along the heart to un-roll the device 600 around the heart. After the device 600 has been deployed, the device 600 is then uncoupled from the elongated delivery device.

Although particular embodiments have been shown and described, it will be understood that they are not intended to limit the claimed inventions, and it will be obvious to those skilled in the art that various changes and modifications may be made without departing from the spirit and scope of the claimed inventions. The specification and drawings are, accordingly, to be regarded in an illustrative rather than restrictive sense. The claimed inventions are intended to cover alternatives, modifications, and equivalents.

1. A method of delivering a material inside a patient, comprising:
inserting a distal end of an elongated device inside a patient next to a heart;
deploying a sheath around at least a part of the heart using the elongated device; and
delivering the material into a space between the heart and the sheath.

2. The method of claim 1, wherein the act of deploying the sheath comprises:
placing the sheath in a rolled-up configuration next to the heart; and
un-rolling the sheath to surround at least a part of the heart.

3. The method of claim 1, wherein the act of deploying the sheath comprises axially moving a member in the elongated device.

4. The method of claim 1, wherein the act of delivering the material comprises delivering the material through an opening at the sheath.

5. The method of claim 1, wherein the material comprises a polymeric material.

6. The method of claim 5, further comprising forming the polymeric material.

7. The method of claim 6, wherein the polymeric material is formed by combining two components to form a cross-linked structure.

8. The method of claim 6, wherein the polymeric material is formed before the material is delivered into the space between the sheath and the heart.

9. The method of claim 6, wherein the polymeric material is formed at the space between the sheath and the heart.

10. The method of claim 1, wherein the material comprises a polyethylene glycol (PEG) based hydrogel.

11. The method of claim 1, wherein the material comprises a 8-arm 10 kDa PEG-vinyl sulfone, and a 4-arm 10 kDa PEG-thiol.

12. The method of claim 1, wherein the material comprises a polymerizing hydrogel.

13. The method of claim 1, wherein the material is biodegradable.

14. The method of claim 1, wherein the material comprises a preformed octomer.

15. The method of claim 1, further comprising removing air pockets between the sheath and the heart using a suction tube.

16. The method of claim 1, wherein the delivered material is in fluid or gel form that is contained by the sheath.

17. A method of preventing heart dilation, comprising:
delivering the material according to the method of claim 1; and
forming a solid layer around at least a part of the heart using the material, wherein the solid layer provides resistance against heart dilation.

18-26. (canceled)

27. A medical device, comprising:
an elongated tube having a proximal end and a distal end, and a body extending between the proximal end and the distal end, wherein the elongated tube further comprises a lumen extending between the proximal end and the distal end;
a deformable sheath coupled to the distal end of the elongated tube;
a member moveable relative to the elongated tube for changing the deformable sheath from a confined configuration to a deployed configuration; and
a delivery lumen located in the member, wherein the delivery lumen is in fluid communication with an opening at the deformable sheath.

28-35. (canceled)

36. The medical device of claim 33, wherein the material comprises a 8-arm 10 kDa PEG-vinyl sulfone, and a 4-arm 10 kDa PEG-thiol.

37-41. (canceled)

42. A medical device, comprising:
an elastic container having a first end, a second end, and a body between the first end and the second end, wherein the first end has an opening, and the second end is closed;
wherein the elastic container has an un-deployed configuration and a deployed configuration; and
wherein when the elastic container is in the deployed configuration, the elastic container has a size and a shape suitable for wrapping around at least a part of a heart.

43-50. (canceled)
